(19) United States
(12) Patent Application Publication
(10) Pub. No.: US 2014/0343382 A1
(43) Pub. Date: Nov. 20, 2014

(54) Methods And Systems For Using A Thermistor In Probe Identification Circuits In Or Associated With Pulse Oximeter Sensors

(71) Applicants: Andrew Kersey, Wallingford, CT (US); David Rich, Glastonbury, CT (US); Dana Rich, Rocky Hill, CT (US)

(72) Inventors: Andrew Kersey, Wallingford, CT (US); David Rich, Glastonbury, CT (US); Dana Rich, Rocky Hill, CT (US)

(73) Assignee: XHALE, INC., Gainesville, FL (US)

(21) Appl. No.: 14/279,372

(22) Filed: May 16, 2014

Related U.S. Application Data
(60) Provisional application No. 61/824,871, filed on May 17, 2013.

(51) Int. Cl. A61B 5/1455; A61B 5/0205

(52) U.S. Cl. CPC A61B 5/14552; A61B 5/0205
USPC 600/324

(57) ABSTRACT

Provided according to embodiments of the present invention are pulse oximetry systems that include a pulse oximeter sensor, and a probe identification circuit that includes a thermistor. The probe identification circuit may be part of or associated with the pulse oximeter sensor.

### CROSS-REFERENCE TO RELATED APPLICATIONS

This application claims the benefit of U.S. Provisional Application No. 61/824,871, filed May 17, 2013, the contents of which are incorporated herein by reference in their entirety.

### BACKGROUND OF THE INVENTION

Photoplethysmography, or “PPG”, is an optical technique for detecting blood volume changes in a tissue. In this technique, one or more emitters are used to direct light at a tissue and one or more detectors are used to detect the light that is transmitted through the tissue (“transmissive PPG”) or reflected by the tissue (“reflectance PPG”). The volume of blood, or perfusion, of the tissue affects the amount of light that is transmitted or reflected. Thus, the PPG signal may vary with changes in the perfusion of the tissue. Information regarding the arterial blood oxygen saturation (SpO₂) of the blood may be obtained by shining red and IR light through the tissue. The amplitude of the pulsatile component of the red and IR light may vary with changes in SpO2 because of the differential absorption of oxygenated and deoxygenated hemoglobin at these two wavelengths. From the amplitude ratio, normalized by the ratio of the amplitudes of the non-pulsatile components, the SpO₂ may be estimated.

Known wavelengths of light are typically needed to illuminate through the tissue of the subject in order to accurately detect the arterial oxygen saturation of a subject. Errors in assumed wavelengths can result in significant errors in the calculation of oxygen saturation, particularly at lower saturations. There is an installed base of existing pulse oximetry probes that use a calibration resistor to allow the monitor to identify the probe type and wavelength parameters of that particular oximetry sensor and thus overcome these potential errors.

### SUMMARY OF THE INVENTION

Provided according to embodiments of the present invention are pulse oximetry systems that include a pulse oximeter sensor, and a probe identification circuit that comprises a thermistor. The probe identification circuit may be part of or associated with the pulse oximeter sensor. For example, in some embodiments, at least part of the probe identification circuit is in an cable connected to the sensor and/or the monitor. In some embodiments of the invention, the probe identification circuit is configured to provide an appropriate identification signal to a monitor so that the pulse oximeter sensor and monitor may operatively connect. In particular embodiments of the invention, the thermistor is in parallel with a standard resistor.

Also provided herein are methods of determining whether a probe identification circuit includes a thermistor. In some embodiments, such methods include transmitting to the probe identification circuit from a medical monitor at least one pulse of current; and detecting with the medical monitor a change in resistance to determine that a thermistor is present in the probe identification circuit. In other embodiments, such methods include detecting with the medical monitor a change in resistance of the probe identification circuit over time due to ambient temperature changes to determine that the thermistor is present in the probe identification circuit.

### BRIEF DESCRIPTION OF THE DRAWINGS

The following drawings are provided to illustrate various aspects of the present inventive concept and are not intended to limit the scope of the present invention unless specified herein.

FIG. 1 is a circuit diagram illustrating an embodiment of the present invention.

FIG. 2 is a graph of resistance as a function of temperature for a thermistor alone and a thermistor with a standard resistor in parallel.

FIG. 3 is a graph of resistance as a function of temperature for a thermistor alone and a thermistor with a standard resistor in parallel.

FIG. 4 is a graph of resistance as a function of temperature for a thermistor alone and a thermistor with a standard resistor in parallel.

### DETAILED DESCRIPTION OF EMBODIMENTS OF THE INVENTION

The present invention now will be described more fully hereinafter with reference to the accompanying drawings, in which embodiments of the invention are shown. However, this invention should not be construed as limited to the embodiments set forth herein. Rather, these embodiments are provided so that this disclosure will be thorough and complete, and will fully convey the scope of the invention to those skilled in the art.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

It will be understood that when an element is referred to as being “on” or “adjacent” to another element, it can be directly on or directly adjacent to the other element or intervening elements may also be present. In contrast, when an element is referred to as being “directly on” or “directly adjacent” to another element, there are no intervening elements present. It will also be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Like numbers refer to like elements throughout the specification.

It will be understood that, although the terms first, second, etc. (or primary, secondary, etc.) may be used herein to describe various elements, these elements should not be limited by these terms. These terms are only used to distinguish one element from another. Thus, a first element discussed below could be termed a second element without departing from the teachings of the present invention.

Traditionally, pulse oximetry probes use a probe identification circuit that includes a calibration resistor to allow a medical monitor to identify the probe type and wavelength parameters of that particular sensor. The medical monitor may determine the resistance in the calibration resistor and calibrate the signals received from the sensor accordingly. In some cases, pulse oximetry probes may be configured to secure to the nose, such as described, for example, in U.S. Publication No. 2014/0005557, incorporated by reference herein in its entirety. Such nasal probes may also include other physiological sensors incorporated into the probe, including, for example, a thermistor for detecting air flow at the nose. The present inventors have discovered a way to incorporate a thermistor, which is also a type of resistor, into a probe identification circuit, so that the same pulse oximetry probe may be used for both oximetry (or other photoplethysmography-based monitoring methods) and thermistor-based respiratory monitoring. Thus, provided according to embodiments of the present invention are devices, systems and methods directed to physiological probes that include a pulse oximetry sensor and a probe identification circuit that includes a thermistor.

As used herein, a “probe identification circuit” refers to a resistor or series of resistors (including a calibration resistor) within and/or associated with the probe that may be used by a medical monitor to identify the wavelength(s) that are being emitted by the light emitting source (e.g., LEDs) and/or other characteristics of the probe.

The terms “medical monitor” or “monitor” refer to one or more processors, generally associated with one or more displays, which receive the signals from a physiological sensor and display data related thereto, such as raw data, processed data, or physiological parameters calculated from the physiological signals.

The term “thermistor” refers to a resistor with a resistance that varies significantly with a change in temperature. In some cases, a thermistor's resistance can vary by a factor over 100 within its stated temperature range. A “standard resistor,” as used herein, refers to a resistor that is not a thermistor.

The term “pulse oximetry sensor” or “sensor”, also referred to as a “pulse oximetry probe” or “probe”, refers generally to any photoplethysmography (PPG) sensor, and the sensor/probe may include other physiological sensors incorporated therein, including a thermistor. The PPG sensor includes one or more components that emit light, and such components will be referred to herein as “emitters.” As used herein, the term “light” is used generically to refer to electromagnetic radiation, and so the term includes, for example, visible, infrared and ultraviolet radiation. Any suitable type of emitter may be used, but in some embodiments, the emitter is a light-emitting diode (LED). In particular embodiments, a first emitter emits light at a first wavelength, and a second emitter light at a second wavelength. For example, a sensor that may be used to measure blood oxygen saturation levels may include a first emitter that emits light in the visible range and a second emitter that emits light in the infrared range. In some cases, a single emitter may emit light at a first wavelength and a second wavelength. One or more photodetectors, also referred to as “detectors”, are also included. The detector is configured to detect light from an emitter, and this detected light generates a PPG signal. Any suitable photodetector may be used. However, examples of photodetectors include photodiodes, photoresistors, phototransistors, light to frequency converters, and the like.

The phrase “associated with the probe” means that the element may not be inside the probe but is in electronic communication with the probe and/or the monitor. For example, one or more of the elements of a probe identification circuit may be present in the cabling or in a device external to the probe but in electronic communication with the probe and/or the monitor. As a particular example, the probe identification circuit may be within the sensor or it may be within a cable (permanent or removeable) in communication with the sensor, and in some cases, part of the probe identification circuit may be within the pulse oximetry sensor and part of the probe identification circuit may be in a cable connected thereto.

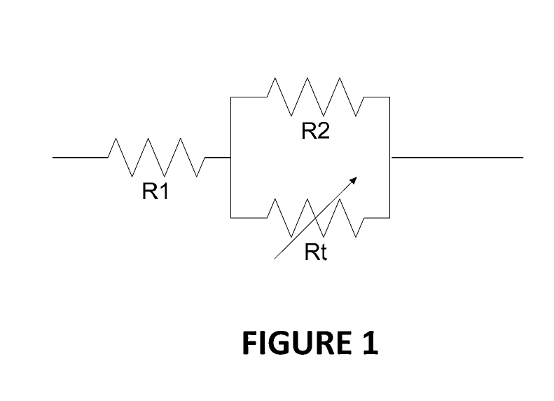

As described above, medical monitors may interface with a calibration resistor to allow the monitor to identify the probe type and wavelength parameters of that particular pulse oximetry sensor. A monitor may also only accept signals from the probe that fall within a particular range of values (the “calibration band”). Thus, in some embodiments, the probe identification circuit is configured so that signals from the probe identification circuit including the thermistor will remain within the desired calibration band regardless of the temperature to which the thermistor is exposed, or at least temperatures to which the thermistor will be exposed (e.g., 0 to 40-50° C.). This may be achieved by any suitable means, but in some cases, the signals from the probe identification circuit/thermistor stay within the calibration band by the use of an additional resistor in parallel with the thermistor. Thus, in some embodiments, provided are sensors having probe identification circuits within or associated therewith that have a resistor in parallel with a thermistor. In some embodiments, the thermistor in parallel with the resistor is in series with the calibration resistor. A particular example of a such an embodiment is shown in the circuit diagram shown in FIG. 1. R1 is the calibration resistor, Rt is the thermistor and R2 is the resistor in parallel with the thermistor.

In some embodiments, the thermistor is configured to be used to detect air flow, but in other embodiments, the thermistor is not detecting air flow and/or the data from the thermistor is not provided to the monitor. For example, in some embodiments, the probe may be connected to a monitor and the monitor may use the calibration circuit to ascertain the wavelength parameters, and no respiration monitoring may be performed. However, in other embodiments, the calibration resistor may not be needed (e.g., because the monitor used is configured specifically for that probe), and the thermistor portion is then used to monitor respiration.

Also provided are devices, systems and methods for ascertaining whether a probe includes a thermistor. Thermistors, when driven with excessive current, create internal heat which can affect the thermistor's performance. For example, the resistance may not be on the correct point of the resistance/temperature curve. This property can be used to detect whether a probe identification circuit includes a thermistor or only standard resistors in circuit. A pure resistor network circuit, while having the same increase in temperature change when driven by excessive current, will not significantly change its overall resistance value, whereas a thermistor in circuit will. Thus, a monitor may provide current to the probe to increase the temperature of the circuit, and assess the change in resistance in order to determine whether the resistor is a non-thermistor resistor or a thermistor.

Thus, for example, when a probe is initially connected to a monitor, a thermistor in circuit can be detected by initially detecting the overall resistance and determining any slow moving baseline change (as only a thermistor will do this), then driving the circuit with a series of short bursts of current pulses to heat the circuit and generate a further change in the measured resistance of the thermistor. Again, a standard resistor in circuit will not change the measured resistance when performing this action, but a thermistor in circuit will.

### Example 1

In this example, a resistor is used in parallel with the thermistor to normalize and/or allow for a more consistent and/or linear change of the thermistor in response to temperature changes over the expected range of exposed temperatures. Referring to the circuit in FIG. 1, the following examples illustrate the use of devices and systems according to particular embodiments of the invention.

A. Selecting the R_{cal-}Compatibility with Existing Pulse Oximeter Calibration Curves:

In order to be compatible with existing pulse oximeter calibration curves, the calibration resistor, thermistor resistor and resistor in parallel may be selected to achieve the desired equivalent resistance (Re). In this example, the Re that correlates to the selected wavelengths is Re=6.65 k. This will be the Rcal value as seen by the monitor.

$$Re = R 1 + ( \frac{Rt * R 2}{Rt + R 2} )$$

$$Rt = 470 @ {25}^{∘} C$$

$$6650 = R 1 + ( \frac{470 * 470}{470 + 470} )$$

$$R 1 = 6415$$

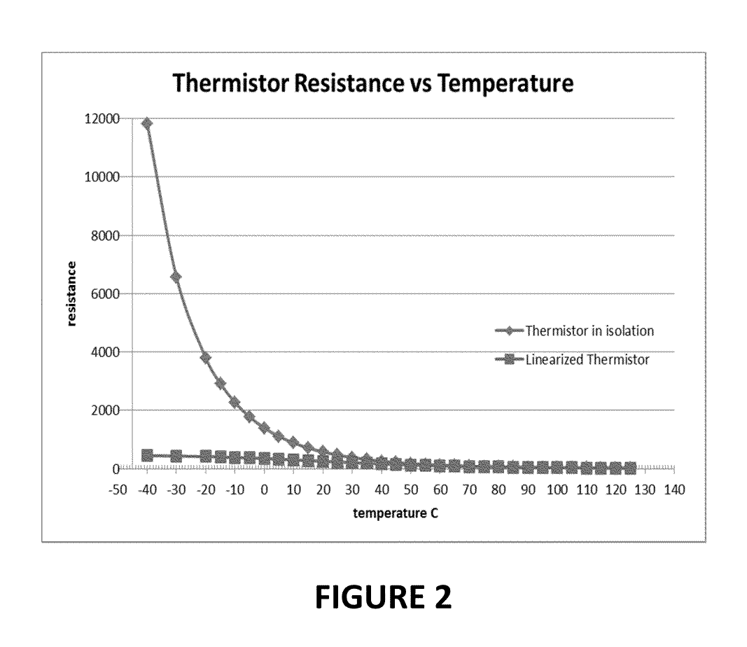

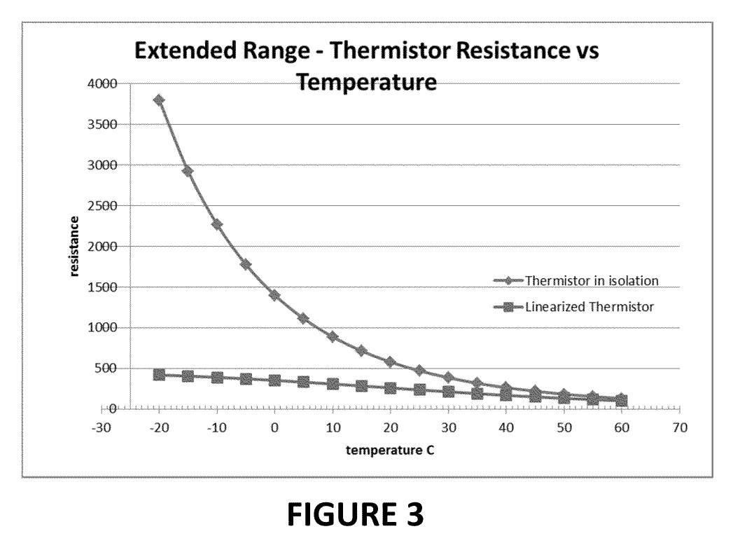

R1 is selected to be 6.42 k, a readily available resistor value. Thus, the appropriate resistors may be selected to provide the desired equivalent resistance to correlate with the appropriate sensor wavelength. FIGS. 2 and 3 illustrated how the resistor in parallel decreases the variation in the resistance of the thermistor over a wide temperature range.

B. Variation Normal Full Scale Swing (0° C.-40° C.)

It is important that the thermistor's change in resistance with temperature does not affect the equivalent resistance to the extent that it will not be recognized by the medical monitor. Thus, the Re may be calculated over a proposed temperature range.

$$Rt = 1399. @ {0}^{∘} C$$

$$Re = 6420 + ( \frac{1399. * 470}{1399. + 470} )$$

$$Re @ {0}^{∘} C = 6771.80$$

$$Rt = 262.2 @ {0}^{∘} C$$

$$Re = 6420 + ( \frac{262.2 * 470}{262.2 + 470} )$$

$$Re @ {40}^{∘} C = 6588.30$$

$$183.50$$

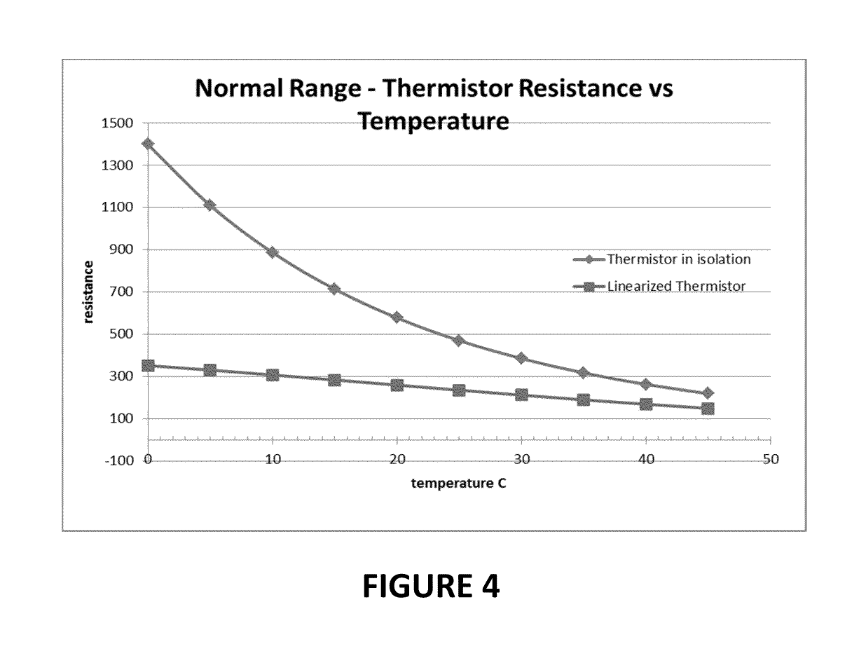

As shown in FIG. 4, the equivalent resistance with the parallel resistor is much more stable over the temperature range than the thermistor resistor alone, although in this case, the thermistor/resistor in parallel combination does decrease somewhat with an increase in temperature.

C. Variation in Resistance for Normal Breath at Room Temperature (24° C.-32° C. @ 23° C. Ambient)

In practice, the temperature variation of a thermistor placed at the nose of a subject will generally not vary to as great of an extent as shown above in Example 1B. Thus, the variation in resistance may be calculated for thermistor temperature variation from the temperature of normal breath to the temperature of room temperature air.

$$Rt = 489.5 . @ {24}^{∘} C$$

$$Re = 6420 + ( \frac{489.5 * 470}{489.5 + 470} )$$

$$Re @ {24}^{∘} C = 6659.78$$

$$Rt = 355.8 @ {32}^{∘} C$$

$$Re = 6420 + ( \frac{355.8 * 470}{355.8 + 470} )$$

$$Re @ {32}^{∘} C = 6622.50$$

$$37.274$$

D. Variation in Resistance for Normal Breath in Cold Room (20° C.-31° C. @ 15° C. Ambient)

The same calculation may be performed to determine the change in resistance of a thermistor as it varies from the temperature of normal breath to the temperature of a cold room.

$$Rt = 577.4 . @ {20}^{∘} C$$

$$Re = 6420 + ( \frac{577.4 * 470}{577.4 + 470} )$$

$$Re @ {20}^{∘} C = 6679.10$$

$$Rt = 370.0 @ {31}^{∘} C$$

$$Re = 6420 + ( \frac{370.0 * 470}{370.0 + 470} )$$

$$Re @ {31}^{∘} C = 6627.02$$

$$52.073$$

Thus, the foregoing examples show that the combination of the thermistor and the resistor in parallel in the probe identification circuit allows for a change in resistance with temperature but not to an extent that prevents the probe from being used with calibration curves available in existing monitors.

In the drawings and specification, there have been disclosed embodiments of the invention and, although specific terms are employed, they are used in a generic and descriptive sense only and not for purposes of limitation, the scope of the invention being set forth in the following claims.

What is claimed:

1. A pulse oximetry system comprising
a pulse oximeter sensor, and
a probe identification circuit that comprises a thermistor,
wherein the probe identification circuit is part of or associated with the pulse oximeter sensor.

2. The pulse oximetry system of claim 1, wherein the probe identification circuit is configured to provide an appropriate identification signal to a monitor so that the pulse oximeter sensor and monitor operatively connect.

3. The probe oximetry system of claim 1, wherein the probe identification circuit is part of the pulse oximeter sensor.

4. The probe oximetry system of claim 1, wherein at least a portion of the probe identification circuit is located in a cable associated with the pulse oximeter sensor.

5. The probe oximetry system of claim 4, wherein the calibration resistor is located in a cable associated with the pulse oximeter sensor and the thermistor is located within the pulse oximeter sensor.

6. The pulse oximetry system of claim 1, further comprising a medical monitor in electronic communication with the pulse oximeter sensor.

7. The pulse oximetry system of claim 6, wherein the equivalent resistance value of the probe identification circuit at a temperature in a range of 0 to 40 degrees C. is a resistance value the monitor will accept for operative connection.

8. The pulse oximetry system of claim 1, wherein the thermistor is in parallel with a standard resistor.

9. The pulse oximetry system of claim 8, wherein the probe identification circuit is configured to provide an appropriate identification signal to a monitor so that the pulse oximeter sensor and monitor operatively connect.

10. The probe oximetry system of claim 8, wherein the probe identification circuit is part of the pulse oximeter sensor.

11. The probe oximetry system of claim 8, wherein at least a portion of the probe identification circuit is located in a cable associated with the pulse oximeter sensor.

12. The probe oximetry system of claim 11, wherein the calibration resistor is located in a cable associated with the pulse oximeter sensor and the thermistor is located within the pulse oximeter sensor.

13. The pulse oximetry system of claim 8, further comprising a medical monitor in electronic communication with the pulse oximeter sensor.

14. The pulse oximetry system of claim 8, wherein the parallel resistor decreases resistance output values for a given thermistor over temperatures in a range of 0 to 40 degrees C.

15. A method of determining whether a probe identification circuit comprises a thermistor comprising
(a) transmitting to the probe identification circuit from a medical monitor at least one pulse of current; and detecting with the medical monitor a change in resistance to determine that a thermistor is present in the probe identification circuit; and/or
(b) detecting with the medical monitor a change in resistance of the probe identification circuit over time due to ambient temperature changes to determine that the thermistor is present in the probe identification circuit.

16. The method of claim 15, wherein the probe identification circuit is part of or associated with a pulse oximeter sensor.

17. The method of claim 16, further comprising monitoring respiration in the individual using the thermistor.

18. The method of claim 16, further comprising monitoring blood oxygen saturation with the pulse oximeter probe.
